(19) United States
(12) Patent Application Publication
(10) Pub. No.: US 2010/0297014 A1
(43) Pub. Date: Nov. 25, 2010

(54) METHOD OF DETECTING THE RISK OF CANCER USING GENETIC MARKERS

(76) Inventor: Paul Jenkins, London (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/682,463

(22) PCT Filed: Oct. 10, 2008
(86) PCT No.: PCT/GB08/03451

§ 371 Date: Jul. 12, 2010

(30) Foreign Application Priority Data
Oct. 10, 2007 (GB) 0719808.8

(51) Int. Cl. A61K 49/00; C12Q 1/68; A61K 31/7088; C07H 21/00; A61P 35/00

(52) U.S. Cl. 424/9.1; 435/6; 514/44 A; 536/24.5

(57) ABSTRACT

An ex vivo method for the detection of the risk of cancer in a patient, comprising the step of:
detecting the expression level of one or more gene sequences identified herein in Table 1, or the complement thereof, or one or more polynucleotides of at least 10 consecutive nucleotides that hybridizes to the one or more sequences (or the complement thereof) under stringent hybridizing conditions, in a sample of genetic material isolated from a patient,
   wherein the expression level indicates the risk of cancer in the patient from whom the sample was isolated.

### FIELD OF THE INVENTION

This invention relates to detecting the risk of cancer, in particular colorectal cancer.

### BACKGROUND TO THE INVENTION

Cancer is the second most common cause of pathological death in developed countries, after cardiovascular disease. Colorectal cancer (CRC) is the second most common cause of cancer death in developed countries, killing 20,000 people a year in the UK.

Screening tests for cancer are being introduced by many health providers, but such tests are not ideal. For example, screening for colorectal cancer usually involves extensive and regular examination of the bowel (colonoscopy) which is uncomfortable, time-consuming, potentially dangerous, has a low pick-up rate and is resource intensive, costing approximately £800. An alternative screening technique for colorectal cancer is the detection of microscopic amounts of blood in the stool, but this is poorly accepted socially, with a low ‘take-up’ rate and leads to many false-positive results, which consequently require colonoscopy.

The use of molecular diagnostics in cancer aims to use predisposition (or predictive) tests to determine genetic susceptibility. Predictive genetic testing refers to the use of a genetic test in an asymptomatic person to create maps of individual risk and predict future risk of disease. The hope underlying such testing is that early identification of individuals at risk of a specific condition will lead to reduced morbidity and mortality through targeted screening, surveillance, and prevention. Consequently, while conventional diagnostic techniques (including radiography and tomography) indicate whether a tumour is already present, tests that identify genetic aberrations are important to indicate the probability of developing a tumour. This knowledge can help devise the best strategy to prevent the development of a tumour.

The identification of reliable genetic markers for cancer is problematic and, to date, no reliable expression signature has been identified that could be used to predict the risk of colorectal cancer in an individual. There is clearly a need for reliable markers for use in predisposition testing for cancer, in particular colorectal cancer.

### SUMMARY OF THE INVENTION

The present invention is based on the surprising identification of a number of genetic markers that are useful in predicting the risk of cancer.

According to a first aspect of the present invention an ex vivo method for the detection of the risk of cancer in a patient comprises the step of detecting the expression level of one or more gene sequences identified herein in Table 1, or the complement thereof, or one or more polynucleotides of at least 10 consecutive nucleotides that hybridises to the one or more sequences (or the complement thereof) under stringent hybridising conditions, in a sample of genetic material isolated from a patient, wherein the expression level indicates the risk of cancer in the patient from whom the sample was isolated.

According to a second aspect of the present invention there is the use of one or more isolated gene sequences identified herein in Table 1, or the complement thereof, or a polynucleotide of at least 10 consecutive nucleotides that hybridises to the one or more sequences (or the complement thereof) under stringent hybridising conditions, in an ex vivo diagnostic assay to test for the risk of cancer in a patient.

According to a third aspect of the invention there is a polynucleotide that hybridises under stringent conditions with and/or alters the expression of an endogenous gene identified in Table 1, for use in therapy. The therapy is preferably the treatment of cancer, more preferably colorectal cancer.

According to a fourth aspect of the present invention there is the use of a polynucleotide that hybridises under stringent conditions with and/or alters the expression of an endogenous gene identified in Table 1, in the manufacture of a medicament for the treatment of cancer, in particular colorectal cancer.

According to a fifth aspect of the invention, a kit for the detection of the risk of cancer in a patient comprises a reagent that binds to a gene identified herein in Table 1, or its complement, or a polynucleotide of at least 10 consecutive nucleotides that hybridises to any of the sequences (or a complement thereof) under stringent hybridising conditions, or a peptide encoded by said gene, complement or fragment, and instructions for detecting the risk of cancer.

According to a sixth aspect of the invention, an in vivo method for the detection of the risk of cancer in a patient comprises the step of detecting the expression level of one or more gene sequences identified herein in Table 1, or the complement thereof, or a polynucleotide of at least 10 consecutive nucleotides that hybridises to the one or more sequences (or the complement thereof) under stringent hybridising conditions, in a patient, wherein the expression level indicates the risk of cancer in the patient.

### DESCRIPTION OF THE INVENTION

The present invention is based on the surprising identification of genes that are effective markers for cancer, in particular colorectal cancer. Identification of the individual genes or their expressed products, such as mRNA or a polypeptide, in a sample obtained from a patient indicates the risk of cancer in the patient. These marker genes are therefore useful in predisposition tests for cancer.

The invention further relates to reagents such as polynucleotide and polypeptide sequences, useful for detecting, diagnosing, monitoring, prognosticating, preventing, imaging, treating or determining a pre-disposition to cancer.

The marker genes identified herein are useful in diagnosing the risk of cancer in a person who has not yet developed the disease, i.e. the marker genes are capable of identifying those individuals who are asymptomatic but who have a genetic predisposition to developing cancer. These individuals would clearly benefit from an early indication of this predisposition as it will allow the regular monitoring of their colorectal tissue, to detect early any potentially cancerous changes.

As used herein, the term “cancer” is to be given its normal meaning in the art, namely a disease characterised by uncontrolled cellular growth and proliferation. The marker genes identified herein are particularly useful in the detection of the risk of colorectal cancer, which is also to be given its usual meaning in the art. For the avoidance of doubt, colorectal cancer refers to cancer that starts in the colon or rectum. The term “colorectal cancer” therefore includes cancers of both the colon and rectum.

The marker genes according to the current invention are detailed in Table 1, below.

TABLE 1

| | | | | |
|---|---|---|---|---|
| | | | Genbank | |
| Sequence | Fold | Common | Accession | |
| Number | change | Name | No. | Description |

Sequences where Upregulation Predicts Risk of Cancer

| | | | | |
|---|---|---|---|---|
| 1 | 23.98 | AQP8 | NM_001169 | aquaporin 8 |
| 2 | 3.08 | AQP7 | NM_001170 | aquaporin 7 |
| 3 | 20.64 | CLCA4 | NM_012128 | chloride channel, |
| | | | | calcium activated, |
| | | | | family member 4 |
| 4 | 20.52 | GUCA2B | NM_007102 | guanylate cyclase |
| | | | | activator 2B |
| | | | | (uroguanylin) |
| 5 | 3.72 | PTPRR | NM_002849 | protein tyrosine |
| | | | | phosphatase, |
| | | | | receptor type, R |
| 6 | 3.34 | CDKN2B | NM_004936 | cyclin-dependent |
| | | | | kinase inhibitor 2B |
| | | | | (p15, inhibits |
| | | | | CDK4) |
| 7 | 7.63 | CA4 | NM_000717 | carbonic |
| | | | | anhydrase IV |
| 8 | 5.01 | CEACAM7 | NM_006890 | carcinoembryonic |
| | | | | antigen-related cell |
| | | | | adhesion molecule |
| | | | | 7 |
| 9 | 4.77 | CEACAM1 | NM_001712 | carcinoembryonic |
| | | | | antigen-related cell |
| | | | | adhesion molecule |
| | | | | 1 (biliary |
| | | | | glycoprotein) |
| 10 | 4.86 | MUCDHL | NM_017717 | mucin and |
| | | | | cadherin-like |
| 11 | 4.79 | CDA | NM_001785 | cytidine deaminase |
| 12 | 3.02 | CHP | NM_007236 | calcium binding |
| | | | | protein P22 |
| 13 | 4.29 | HPGD | NM_000860 | hydroxyprostaglan |
| | | | | din dehydrogenase |
| | | | | 15-(NAD) |
| 14 | 3.73 | MAD | NM_002357 | MAX dimerization |
| | | | | protein 1 |
| 15 | 3.04 | ADAM8 | NM_001109 | a disintegrin and |
| | | | | metalloproteinase |
| | | | | domain 8 |
| 16 | 3.49 | DSC2 | NM_024422 | desmocollin 2 |

Sequences where Downregulation Predicts Risk of Cancer

| | | | | |
|---|---|---|---|---|
| 17 | 0.12 | HOXD13 | NM_000523 | homeo box D13 |
| 18 | 0.13 | TFPI2 | NM_006528 | tissue factor |
| | | | | pathway inhibitor 2 |
| 19 | 0.22 | RBMS1 | NM_016837 | RNA binding motif, |
| | | | | single stranded |
| | | | | interacting protein |
| | | | | 1 |
| 20 | 0.24 | RAB23 | NM_016277 | RAB23, member |
| | | | | RAS oncogene |
| | | | | family |
| 21 | 0.28 | P164RHOGE | NM_014786 | Rho-specific |
| | | F | | guanine-nucleotide |
| | | | | exchange factor 164 |
| | | | | kDa |
| 22 | 0.31 | MMP19 | NM_002429 | matrix |
| | | | | metalloproteinase |
| | | | | 19 |
| 23 | 0.35 | MMP3 | NM_002422 | matrix |
| | | | | metalloproteinase 3 |
| | | | | (stromelysin 1, |
| | | | | progelatinase) |
| 24 | 0.37 | CALCRL | NM_005795 | calcitonin receptor- |
| | | | | like |
| 25 | 0.18 | COL4A1 | NM_001845 | collagen, type IV, |
| | | | | alpha 1 |
| 26 | 0.19 | DES | NM_001927 | desmin |
| 27 | 0.25 | CNN1 | NM_001299 | calponin 1, basic, |
| | | | | smooth muscle |
| 28 | 0.26 | MFAP4 | NM_002404 | microfibrillar- |
| | | | | associated protein 4 |
| 29 | 0.27 | ELN | NM_000501 | elastin |
| | | | | (supravalvular aortic |
| | | | | stenosis, Williams- |
| | | | | Beuren syndrome) |
| 30 | 0.27 | LMOD1 | NM_012134 | leiomodin 1 (smooth |
| | | | | muscle) |
| 31 | 0.30 | EMILIN | NM_007046 | elastin microfibril |
| | | | | interface located |
| | | | | protein |
| 32 | 0.31 | VCL | NM_0033373 | vinculin |
| 33 | 0.23 | PTGS2 | NM_000963 | prostaglandin- |
| | | | | endoperoxide |
| | | | | synthase 2 |
| | | | | (prostaglandin G/H |
| | | | | synthase and |
| | | | | cyclooxygenase) |
| 34 | 0.42 | IGFBP7 | NM_001553 | insulin-like growth |
| | | | | factor binding |
| | | | | protein 7 |
| 35 | 0.35 | IGFBP5 | NM_000599 | insulin-like growth |
| | | | | factor binding |
| | | | | protein 5 |
| 36 | 0.33 | RGS1 | NM_002922 | regulator of G- |
| | | | | protein signalling 1 |
| 37 | 0.30 | SOCS3 | NM_003955 | suppressor of |
| | | | | cytokine signaling 3 |
| 38 | 9.3 | IGFBP3 | NM_0010133 | insulin-like growth |
| | | | 98 (Variant 1) | factor binding |
| | | | NM_000598 | protein 3 |
| | | | (Variant 2) | |
| 39 | 3.19 | Cyp27B | NM_000785. | P450 |
| | | | 3 | monooxygenase |
| 40 | 1.69 | Cox-1 | NM_000962 | prostaglandin- |
| | | | (Variant 1) | endoperoxide |
| | | | NM_080591 | synthase |
| | | | (Variant 2) | |
| 41 | 5.43 | IGF-1R | NM_000875 | Insulin-like growth- |
| | | | | factor-1 receptor |

These marker genes were identified by comparing gene expression patterns between colorectal tissue obtained from normal, non-cancer patients and the “normal” (i.e. non-cancerous) tissue adjacent to cancerous colorectal tissue. This methodology has identified genes that indicate a predisposition to cancer, in particular colorectal cancer.

Diagnosis can be made on the basis of the presence, absence or extent of expression of the gene or gene product in the patient. As used herein, the term “gene product” refers to the mRNA or polypeptide product that results from transcription and/or translation of the gene. The methods to carry out the diagnosis can involve the synthesis of cDNA from the mRNA in a test sample, amplifying as appropriate, portions of the cDNA corresponding to the genes or fragments thereof and detecting each product as an indication of the risk of the disease in that tissue, or detecting translation products of the mRNAs comprising gene sequences as an indication of the risk of the disease.

The presence, absence or level of expression of the gene or gene product in the patient can be detected in vivo or ex vivo. In a preferred embodiment, expression is detected in vitro, in a sample of genetic material that is isolated from the patient. The sample material is preferably isolated from colorectal tissue. As the gene or gene product is useful as a marker for the risk of cancer, it is preferred that the tissue sample is not already cancerous. Therefore, a preferred tissue sample is from non-cancerous colorectal tissue. The tissue may be obtained by any suitable means, for example by biopsy. Alternatively, expression of the marker gene can be carried out in vivo, for example using techniques such as “Quantum Dot” labelling.

Highly luminescent “Quantum Dots”, which are known in the art, are highly stable against photo-bleaching and have narrow, symmetric emission spectra. The emission wavelength of quantum dots can be continuously tuned by changing the particle size or composition, and a single light source can be used for simultaneous excitation of all different-coloured dots. Bioconjugated quantum dots typically comprise a collection of different sized nanoparticles embedded in tiny beads of polymer material. These can be finely tuned to various luminescent colours that can be used to label one or more sequences that hybridise to genes identified herein as predictive for cancer risk. The quantum dot labelled sequences can be targeted to the colon or rectum using techniques known to the skilled person, for example using an antibody that is specific to a protein that is expressed in the colorectal tissue. For example, a conjugated anti-guanylyl cyclase C receptor antibody will target the quantum dot-labelled sequences to the colon following injection into the bloodstream. A number of other techniques for delivering quantum dot labelled marker sequences to colorectal cells will be apparent to the skilled person, including the use of translocation peptides, liposomes and endocytic uptake. One preferred system is based on the use of small cyclic repeating molecules of glucose known as cyclodextrins, that are assembled into linear cyclodextrin-containing polymers. These can be synthesised over a broad range of molecular weights, providing tuneable properties for marker delivery that improve localisation at the target tissue. Another preferred approach coats quantum dots with a polymer such as poly(ethylene glycol) (PEG), and attaches these coated dots to a homing peptide (e.g. guanylyl cyclase c receptor) and one or more specific markers targeting a gene identified in Table 1 as predisposing to cancer, thereby forming a nanoparticle. As binding to the target (colorectal) tissue occurs, the nanoparticle is taken up by the colonic cells and the oligonucleotide probes bind to their target complementary RNA. Since each marker is associated with a specific quantum dot emitting fluorescence at a specific wavelength, both intensity and spectrum of emission are indicative of successful hybridisation and presence of target mRNA.

If the individual's colon expresses the specific gene(s) to which a marker-quantum dot conjugate is complementary, the quantum dots will hybridise to their targets within the colon and emit light at a characteristic wavelength. This will result in a colour signal for real-time “optical biopsy”. The quantum dots can be detected by infra-red optical imaging in vivo, for example in the colon, directly through the tissue or by using a colonoscope allowing a real-time optical “biopsy”. This procedure would result in a diagnosis without tissue removal. This technique can also be used to monitor a diagnosis or treatment.

In a preferred embodiment, a plurality of the marker sequences disclosed herein are identified, either sequentially or simultaneously, in a sample or samples obtained from a patient in order to diagnose the risk of cancer. In a preferred embodiment, two, three, four, five or more, for example ten or more, marker sequences are detected. In a further preferred embodiment, at least one upregulated marker (selected from sequences 1 to 16) and at least one downregulated marker (selected from sequences 17 to 41) is detected; preferably, two, three, four, five or more upregulated and two, three, four, five or more downregulated markers are detected.

The present invention is also concerned with the use of isolated polynucleotides that comprise the sequences of the genes identified in Table 1, their complements, or fragments thereof that comprise at least 10 consecutive nucleotides, preferably at least 15 consecutive nucleotides, more preferably 30 nucleotides, yet more preferably at least 50 nucleotides. Polynucleotides that hybridise to a polynucleotide as defined above, are also within the scope of the invention. Hybridisation will usually be carried out under stringent conditions, known to those in the art, chosen to reduce the possibility of non-complementary hybridisation. Examples of suitable hybridising conditions are disclosed in Nucleic Acid Hybridisation: A Practical Approach (B. D. Hames and S. J. Higgins, editors IRL Press, 1985). An example of stringent hybridisation conditions is overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulphate, and 20 μg/ml denatured, sheared salmon sperm DNA, followed by washing in 0.1×SSC at about 65° C.

The use of isolated peptides encoded by the genes identified in Table 1 is within the scope of the invention. Preferably a peptide comprises any of the sequences encoded by the genes of Table 1 or a fragment thereof of at least 10 amino acid residues.

Homologues of the genes identified in Table 1 are within the scope of the invention. The term “homologue” refers to a sequence that is similar but not identical to one of the identified genes. A homologue performs the same function as the identified gene, i.e. the same biological function. The common name, Genbank accession number and description of each marker sequence is provided in Table 1; a homologue of a marker sequence according to the invention must retain the biological function of the sequence. The biological function of each sequence in Table 1 is known, and is summarised in the “description” column of Table 1. For example, a homologue of AQP8 must retain Aquaporin 8 function, which is known to be a membrane water channel.

Whether two sequences are homologous is routinely calculated using a percentage similarity or identity, terms that are well known in the art. Homologues preferably have 70% or greater similarity or identity at the nucleic acid or amino acid level, more preferably 80% or greater, more preferably 90% or greater, such as 95% or 99% identity or similarity at the nucleic acid or amino acid level. A number of programs are available to calculate similarity or identity; preferred programs are the BLASTn, BLASTp and BLASTx programs, run with default parameters, available at www.ncbi.nlm.nih.gov. For example, 2 nucleotide sequences may be compared using the BLASTn program with default parameters (score=100, word length=11, expectation value=11, low complexity filtering=on). The above levels of homology are calculated using these default parameters.

The skilled person will realise that a gene or gene product identified in a patient may differ slightly from the exact gene or product sequence provided herein, yet is still recognisable as the same gene or gene product. Any gene or gene product that is recognisable by a skilled person as the same as one referred to herein, is within the scope of the invention. For example, a skilled person may identify a polynucleotide or polypeptide under investigation by a partial sequence and/or a physical characteristic, such as the molecular weight of the gene product. The gene or gene product in a patient may be an isoform of that defined herein. Accordingly, isoforms and splice variants are within the scope of the present invention. The skilled person will realise that differences in sequences between individuals, for example single nucleotide poymorphisms, are within the scope of the invention. The key to the invention is that the polynucleotide or polypeptide that is identified in a sample isolated from a patient is recognisable as one characterised herein.

Useful reagents include polynucleotides comprising the isolated gene sequences described in Table 1, their complements, or fragment(s) thereof which may be useful in diagnostic methods such as RT-PCR, PCR or hybridisation assays of mRNA extracted from biopsied tissue, blood or other test samples; or proteins which are the translation products of such mRNAs; or antibodies directed against these proteins. Any of these useful reagents may be incorporated into a kit that can be used to diagnose the risk of cancer. Labels, such as quantum dots or a fluorescent label, which may be useful in an assay may also be incorporated into the kit. These assays also include methods for detecting the gene products (proteins) in light of possible post-translational modifications that can occur in the body, including interactions with molecules such as co-factors, inhibitors, activators and other proteins in the formation of sub-unit complexes.

How each gene is differentially expressed in patients at risk of cancer is indicated in Table 1. For those genes that show increased expression in individuals with a predisposition for cancer (i.e. genes that are “upregulated” in predisposed individuals), an increased level of a gene product in a sample isolated from a patient is indicative of the risk of cancer. For those genes that show decreased expression in individuals with a predisposition for cancer (i.e. genes that are “downregulated” in predisposed individuals), a decreased level of a gene product in a sample isolated from a patient is indicative of the risk of cancer. As used herein, the terms “upregulated” and “downregulated” preferably refer to a significant change in the level of expression compared to the control. Significant levels will be apparent to the skilled person; preferably a three-fold change in expression is observed.

For the avoidance of doubt, the genes identified in Table 1 as sequence numbers 1 to 16 show increased expression in individuals at risk of cancer and the genes identified in Table 1 as sequence numbers 17 to 41 show decreased expression in individuals at risk of cancer.

The skilled person will understand that the terms “increased” and “decreased” refer to the amount of a gene product in a sample, compared to a “control” sample, or a known level of expression, that is indicative of a “healthy” patient that is not predisposed to cancer. For example, in the embodiment wherein the sample is isolated from the normal colorectal tissue of a patient, the expression level in this sample is compared to a “control” sample of normal, non-cancer colorectal tissue from a non-cancer patient or known level of expression for normal, non-cancer colorectal tissue from a non-cancer patient.

In an alternative embodiment, the amount of gene product in a sample can be compared to a “control” sample or known level of expression that is indicative of a patient that is known to be predisposed to cancer.

Identification of the genes or their expressed products may be carried out by techniques known for the detection or characterisation of polynucleotides or polypeptides. For example, isolated genetic material from a patient can be probed using short oligonucleotides that hybridise specifically to the target gene. The oligonucleotide probes may be detectably labelled, for example with a fluorophore, so that upon hybridisation with the target gene, the probes can be detected. Alternatively, the gene, or parts thereof, may be amplified using the polymerase enzyme, e.g. in the polymerase chain reaction, with the amplified products being identified, again using labelled oligonucleotides.

Diagnostic assays incorporating any of the genes, proteins or antibodies according to the invention will include, but are not limited to:
Polymerase chain reaction (PCR)
Reverse transcription PCR
Real-time PCR
In-situ hybridisation
Southern dot blots
Immuno-histochemistry
Ribonuclease protection assay
cDNA array techniques
ELISA
Protein, antigen or antibody arrays on solid supports such as glass or ceramics.
Small interfering RNA functional assays.

All of the above techniques are well known to those in the art. Preferably, the diagnostic assay is carried out in vitro, outside of the body of the patient.

The preferred diagnostic technique is Real-time PCT. Real-time PCR, also known as kinetic PCR, qPCR, qRT-PCR and RT-qPCR, is a quantitative PCR method for the determination of copy numbers of templates such as DNA or RNA in a PCR reaction. There are two kinds of Real-time PCR: probe-based and intercalator-based. Both methods require a special thermocycler equipped with a sensitive camera that monitors the fluorescence in each reaction at frequent intervals during the PCR reaction. Probe-based real-time PCR, also known as TaqMan PCR, requires a pair of PCR primers (as in regular PCR) and an additional fluorogenic probe which is an oligonucleotide with both a reporter fluorescent dye and a quencher dye attached. The intercalator-based method, also known as the SYBR Green method, requires a double-stranded DNA dye in the PCR reaction which binds to newly synthesised double-stranded DNA and gives fluorescence.

The identification of the genes in Table 1 also permits therapies to be developed, with each gene being a target for therapeutic molecules. For example, there are now many known molecules that have been developed for gene therapy, to target and prevent the expression of a specific gene. Molecules of particular interest are small interfering RNA (siRNA) molecules and micro RNA (miRNA) molecules. Small interfering RNA (siRNA) suppresses the expression of a specific target protein by stimulating the degradation of the target mRNA. Micro RNA's (miRNA's) are single stranded RNA molecules of about 20 to 25, usually 21 to 23, nucleotides that are thought to regulate gene expression. Other synthetic oligonucleotides are also known which can bind to a gene of interest (or its regulatory elements) to modify expression. Peptide nucleic acids (PNAs) in association with DNA (PNA-DNA chimeras) have also been shown to exhibit strong decoy activity, to alter the expression of the gene of interest. Molecules, preferably polynucleotides, that can alter the expression level of a gene identified in Table 1 are therefore useful in the prevention and treatment of cancer, preferably colorectal cancer, and are within the scope of the invention. The skilled person will realise whether up-regulation or down-regulation (inhibition) of each gene is required.

The present invention also includes antibodies raised against a peptide of any of the genes identified in the invention. The term “antibody” refers broadly to any immunologic binding agent such as IgG, IgM, IgA, IgD and IgE. An antibody binds, preferably specifically, to an antigen. Antibody is also used to refer to any antibody-like molecule that has an antigen-binding region and includes antibody fragments such as single domain antibodies (DABS), Fv, scFv, aptamers, etc. The techniques for preparing and using various antibody-based constructs and fragments are well known in the art. Means for preparing and characterising antibodies are also well known in the art.

The antibodies will usually have an affinity for the peptide, encoded by a gene identified in Table 1, of at least 10⁻⁶M, more preferably, 10⁻⁹M and most preferably at least 10⁻¹¹M. The antibody is preferably specific to the peptide of the invention, i.e. it binds with high affinity only to a specific peptide of the invention, and does not bind to other peptides. This allows the antibody to bind specifically to the peptide of the invention in a mixture containing a number of different peptides. The antibody may be of any suitable type, including monoclonal or polyclonal. Combinations of antibodies to two, three, four, five or more peptides encoded by genes according to Table 1 are within the scope of the invention.

Assay kits for determining the presence of the peptide antigen in a test sample are also included. In one embodiment, the assay kit comprises a container comprising an antibody that specifically binds to the antigen, wherein the antigen comprises at least one epitope encoded by a gene identified in Table 1. The kit can contain antibodies to epitopes encoded by multiple genes according to Table 1; the different antibodies can be packaged together (in a single container), or separately, within the kit. These kits can further comprise containers with useful tools for collecting test samples, such as blood, saliva, urine and stool. Such tools include lancets and absorbent paper or cloth for collecting and stabilising blood, swabs for collecting and stabilising saliva, cups for collecting and stabilising urine and stool samples. The antibody can be attached to a solid phase, such as glass or a ceramic surface.

Detection of antibodies that bind specifically to any of the antigens in a test sample suspected of containing these antibodies may also be carried out. This detection method comprises contacting the test sample with a polypeptide, which contains at least one epitope of a gene identified in Table 1. Contact is performed for a time and under conditions sufficient to allow antigen/antibody complexes to form. The method further entails detecting complexes, which contain any of the polypeptides. The polypeptide complex can be produced recombinantly or synthetically or be purified from natural sources.

In a separate embodiment of the invention, antibodies, or fragments thereof, against any of the antigens can be used for the detection of the location of the antigen in a patient for the purpose of detecting or diagnosing the disease or condition. Such antibodies can be monoclonal or polyclonal, or made by molecular biology techniques and can be labelled with a variety of detectable agents, including, but not limited to radioisotopes.

In a further embodiment of the invention, antibodies or fragments thereof, whether monoclonal or polyclonal or made by molecular biology techniques, can be used as therapeutics for the disease characterised by the expression of any of the genes of the invention. The antibody may be used without derivatisation, or it may be derivatised with a cytotoxic agent such as radioisotope, enzyme, toxin, drug, pro-drug or the like.

If desired, the cancer screening methods of the present invention may be readily combined with other methods in order to provide an even more reliable indication of diagnosis or prognosis, thus providing a multi-marker test.

1. An ex vivo method for the detection of the risk of cancer in a patient, comprising the step of:
(i) detecting the expression level of a gene sequence of prostaglandin-endoperoxide synthase 2 prostaglandin G/H synthase and cyclooxygenase), or the complement thereof, or one or more polynucleotides of at least 10 consecutive nucleotides that hybridizes to the gene sequence (or the complement thereof) under stringent hybridizing conditions, in a sample of genetic material isolated from a patient,
wherein the expression level indicates the risk of cancer in the patient from whom the sample was isolated.

2. The method according to claim 1, wherein the sample of genetic material is isolated from non-cancerous colorectal tissue.

3. The method according to claim 1, wherein the cancer is colorectal cancer.

4-5. (canceled)

6. A method for treating cancer in a subject, comprising administering a polynucleotide that hybridizes under stringent conditions with, and/or alters the expression of, an endogenous prostaglandin-endoperoxide synthase 2 (prostaglandin G/H synthase and cyclooxygenase) gene to the subject.

7. A medicament comprising a polynucleotide that hybridizes under stringent conditions with, and/or alters the expression of, an endogenous prostaglandin-endoperoxide synthase 2 (prostaglandin G/H synthase and cyclooxygenase) gene.

8. A kit for the detection of the risk of cancer in a patient, comprising a reagent that binds to a prostaglandin-endoperoxide synthase 2 (prostaglandin G/H synthase and cyclooxygenase) gene sequence, or its complement, or a polynucleotide of at least 10 consecutive nucleotides that hybridizes to the sequence (or a complement thereof) under stringent hybridizing conditions, or a peptide encoded by said gene, complement or fragment, and instructions for detecting the risk of cancer.

9. The kit according to claim 8, wherein the reagent is an antibody that binds to a peptide encoded by said gene.

10. The kit according to claim 8, wherein the reagent is a polynucleotide that hybridises to said gene.

11. The kit according to claim 8, further comprising quantum dots.

12. An in vivo method for the detection of the risk of cancer in a patient, comprising the step of:
(i) detecting the expression level of a gene sequence of prostaglandin-endoperoxide synthase 2 (prostaglandin G/H synthase and cyclooxygenase), or the complement thereof, or a polynucleotide of at least 10 consecutive nucleotides that hybridises to any of the one or more sequences (or the complement thereof) under stringent hybridising conditions, in a patient wherein the expression level indicates the risk of cancer in the patient.

13. The method according to claim 12, wherein the cancer is colorectal cancer.

14. The method according to claim 6, wherein the cancer is colorectal cancer.

15. The method according to claim 1, wherein the gene sequence of prostaglandin-endoperoxide synthase 2 is that identified by Genbank Accession Number NM_{—}000963.

16. The method according to claim 6, wherein the gene of prostaglandin-endoperoxide synthase 2 is that identified by Genbank Accession Number NM_{—}000963.

17. The medicament according to claim 7, wherein the gene of prostaglandin-endoperoxide synthase 2 is that identified by Genbank Accession Number NM_{—}000963.

18. The kit according to claim 8, wherein the gene sequence of prostaglandin-endoperoxide synthase 2 is that identified by Genbank Accession Number NM_{—}000963.

19. The method according to claim 12, wherein the gene sequence of prostaglandin-endoperoxide synthase 2 is that identified by Genbank Accession Number NM_{—}000963.
